(19) United States
(12) Patent Application Publication
(10) Pub. No.: US 2010/0022520 A1
(43) Pub. Date: Jan. 28, 2010

(54) MALONYL-COA ACETYLTRANSFERASE INHIBITORS AGAINST ANTIBIOTIC RESISTANT BACTERTIA

(75) Inventor: Alexander Doemling, Pittsburgh, PA (US)

Correspondence Address: FOLEY AND LARDNER LLP;SUITE 500, 3000 K STREET NW, WASHINGTON, DC, 20007, US

(73) Assignee: University of Pittsburgh -- Of the Commonwealth System of Higher Education

(21) Appl. No.: 12/470,958

(22) Filed: May 22, 2009

Related U.S. Application Data
(60) Provisional application No. 61/055,982, filed on May 24, 2008.

(51) Int. Cl. A61K 31/381; A61K 31/542; A61K 31/54; A61K 31/541; A61K 31/506; A61K 31/41; A61P 39/06; A61K 31/505; A61P 3/04

(52) U.S. Cl. 514/224.2; 514/227.5; 514/227.8; 514/274; 514/382; 514/445

(57) ABSTRACT

Compounds characterized by thiolactone or thiomorpholino cores display useful antibiotic and radioprotective properties. The latter properties are believed to arise from an ability of the compounds to inhibit proteins involved in apoptosis.

### CROSS-REFERENCE TO RELATED PATENT APPLICATIONS

This application claims priority from U.S. provisional application Ser. No. 61/055,982, filed May 24, 2008, which is incorporated herein by reference in its entirety.

### BACKGROUND OF THE INVENTION

Bacteria and other microorganisms that cause infections are remarkably resilient and can develop ways to survive drugs meant to kill or weaken them. This antibiotic resistance, also known as antimicrobial resistance or drug resistance, is due largely to the increasing use of antibiotics. After several decades of widespread use of antibiotics, the effectiveness of these drugs has eroded and in recent years is emerging as a serious medical problem worldwide. Indeed, by now hospital and community acquired infections with multiple drug-resistant microorganisms pose a considerable threat to society. According to recent statistics, in the United States alone nearly two million patients contract bacterial infection in the hospital each year, of these over 100,000 die as a result of their infection (up from 13,300 patient deaths in 1992). Furthermore, about 70% of the bacteria that cause hospital-acquired infections are resistant to at least one of the drugs most commonly used to treat them. For example, in the average hospital about 60% of S. aureus isolates are methicillin-resistant. The corresponding financial consequences arising for the treatment of infections arising from antibiotic resistant bacteria is staggering, with billions spent in the United States alone.

In addition to obvious public health initiatives, such as curtailing the inappropriate use of antibiotics for viral infections, there is a clear medical need to develop a new line of pharmaceutical agents to combat antibiotic resistant bacterial strains. In this regard, the enzymes important for bacterial fatty acid synthesis, provide promising targets for the development of new antimicrobial agents, especially, in view of the fact that the bacterial enzymes are fundamentally different from their human and mammalian counterparts. Moreover, studies involving gene deletions and mutations have revealed that the enzymes responsible for bacterial fatty acid synthesis are crucial for bacterial cell survival.

The thiolactone heterocyclic scaffold (Scheme 1), is found in a variety of microbial natural products. Compounds within this scaffold exhibit potent antibacterial and antitumor activities, such as thiolactomycin 1. Sasaki, H. et al., J. Antibiot. 1982, 35, 396-400. Additionally, Erdosteine® 2 has been approved by the U.S. FDA for the treatment of chronic obstructive lung disease, and Prasugel® 3 is under development as a potent inhibitor of platelet aggregation in vivo. See Dal Negro, et al., Pulmonary Pharmacology & Therapeutics, 2008, 21(2), 304-308 and Farid, Nagy A., et al., Drug Metabolism and Disposition 2007, 35(7), 1096-1104.

The thiolactone core that is common to these natural products (see Scheme 1) also is widely used as a synthetic intermediate for the construction of compounds with applications in catalysis and in medicinal chemistry. In addition, bioactive compounds having the thiolactone scaffold have been used as candidate therapeutics for treating Alzheimer's disease, for modulating H3 activity, and for anticancer purposes.

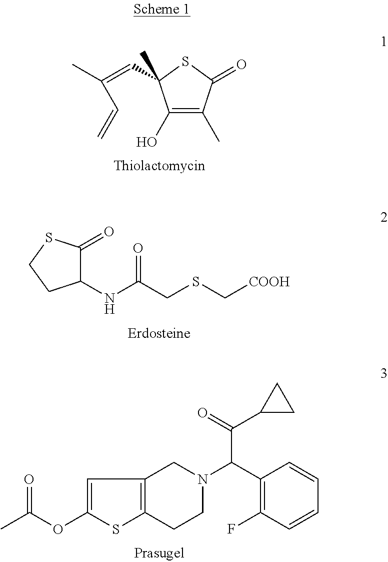

Similar to the thiolactone scaffold, the 2-keto thiomorpholine core is important in medicinal chemistry, and several compounds having this core are described in the literature as antibacterial topoisomerase-IV inhibitors. See PCT application WO 2003064421 (2003) to Miller et al.

### SUMMARY OF THE INVENTION

The present invention concerns the synthesis of various compounds that have a thiolactone or a thiomorpholino core. Formulations of the inventive compounds are useful as antibiotics. Accordingly, the invention provides an approach for treating bacterial infection by administering a formulation that includes a therapeutic dose of a compound according to one of Formula I, Formula II or Formula III

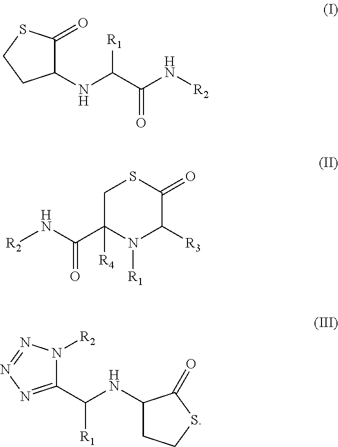

For compounds of Formulae I-III, R₁ is selected from the group consisting of (C₁-C₆) alkyl, (C₃-C₆)aryl, and (C₃-C₆)heteroaryl, arylakyl, and heteroarylalkyl. Substituent R₂ is selected from the group consisting of (C₁-C₆)alkyl, (C₃-C₆)aryl, (C₃-C₆)heteroaryl, arylakyl, heterocycloalkyl, and heteroarylalkyl. Substituent R₃ is selected from the group consisting of (C₁-C₆)alkyl, (C₃-C₆)aryl, and (C₃-C₆)heteroaryl, arylakyl, heterocycloalkyl, and heteroarylalkyl and substituent R₄ is selected from the group consisting of (C₁-C₆) alkyl, and heterocycle.

From an anticancer perspective, compounds of the invention also can prevent the toxic side effects that accompany radiation chemotherapy. Thus, a methodology is provided for protecting normal tissue in a subject undergoing radiation chemotherapy by administering to the subject a therapeutic dose of a compound according to any one of Formulae I-III, supra.

The invention also encompasses pharmaceutical compositions comprised of compounds that conform to one of the above-mentioned formulae. In this vein, the invention also provides pharmaceutically acceptable salts, solvates, stereoisomers, tautomers, and prodrugs of such compounds, which likewise can be formulated with a pharmaceutically acceptable carrier.

### BRIEF DESCRIPTION OF THE DRAWINGS

FIG. 1 graphically depicts a structure, elucidated via x-ray crystallography, of the malonyl-CoA-FabD complex (pdb 2G2Z). As shown in this graphical depiction, FabD does not bind malonyl-CoA. Rather, malonyl-CoA is cleaved by an active site serine resulting in the formation of a covalently adduct. CoA released during the cleavage reaction remains bound to the enzyme, electrostatically. Top: CoA (pink) bound to the surface channel of FabD in front of the hidden active site grove. Noteworthy are the multiple hydrogen bonds (yellow dotted), which an inhibitor preferably would mimic. Bottom: Close-up view of the active site, with malonyl covalently bound to the S⁹² residue of FabD. Of importance is the salt bridge between R¹¹⁷ and the terminal carboxyl group of malonate. This interaction is necessary for the proper orientation of malonate in the active site; hence, inhibitors of FabD preferably would mimic such an interaction.

FIG. 2 graphically depicts survival curves for 32D cl 3 mouse hematopoietic progenitor cells treated with inventive p53 inhibitors BEB 55, BEB 59 and BEB 69 prior to exposing the cells radiation.

FIG. 3 graphically depicts survival curves for 32D cl 3 mouse hematopoietic progenitor cells treated with inventive compounds BEB 75, SK-7 and SS61 prior to their exposure to radiation.

FIG. 4 graphically depicts survival curves for 32D cl 3 mouse hematopoietic progenitor cells that were treated with the inventive compounds after exposing the cells to radiation.

FIG. 5 schematically shows the FabD catalyzed transfer of malonate to holo-acyl carrier protein during fatty acid synthesis in cells. Also depicted is an in vitro enzyme assay for measuring the percent inhibition of FabD in the presence of an inhibitor for this enzyme. The percent inhibition of FabD is calculated by analyzing the reaction mixture using high-performance liquid chromatography and integrating the area under the CoA peak in a chromatogram.

### DETAILED DESCRIPTION OF THE PREFERRED EMBODIMENTS

The present invention focuses, respectively, on inhibiting the enzyme malonyl-CoA acetyltransferase (MCAT), also known as “FabD,” and on ameliorating or preventing apotosis of cells after exposure to ionizing radiation.

The protein FabD is encoded by the gene fabD. FabD catalyzes the last reaction of the initiation steps of fatty acid synthesis in all bacteria, namely, FabD serves to transfer malonate from malonyl-CoA to the terminal thiol of holo-acyl carrier protein (ACP).

Support for the role of FabD as a suitable target for the development of new broad-spectrum antibiotic agents stems from the discovery that the deletion of the gene for this enzyme is lethal for a representative range of bacteria studied by the present inventor. Thus, compounds that are inhibitors of MCAT are promising candidate therapeutics for treating bacterial infections. In this context, the term “antibiotic” refers to a substance or compound (also called chemotherapeutic agent) that kills or inhibits the growth of bacteria.

Inhibitors of MCAT, were designed using a structure-based approach. To facilitate inhibitor design, sequence analysis of FabD from E. coli, S. aureus, and other prokaryotes was carried out and revealed a high homology for this enzyme within the microorganisms analyzed, especially within the active site region of this protein.

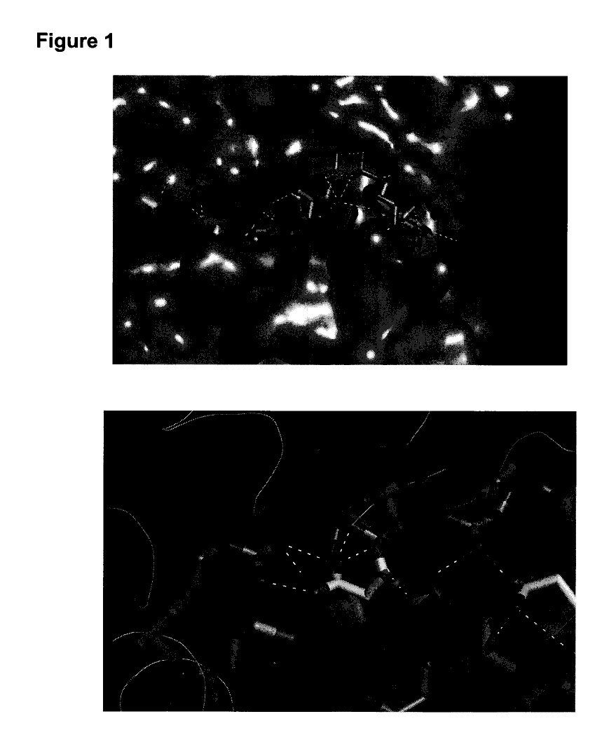

FIG. 1 shows the structure of E. coli FabD bound to malonyl CoA. See Oefner, C. et al., Acta Crystal. Acta D 62, 2006, 613-618. Analysis of the x-ray coordinates reveal that the malonyl moiety of the malonyl CoA complex is cleaved and covalently bound to an active site serine (Ser-92), whereas the CoA group resides in an active site groove, and is electrostatically bound to several active site residues. Also seen in FIG. 1 are multiple hydrogen bonds (dotted yellow lines) between CoA and the active site, as well as a crucial salt bridge between the carboxylate group of malonate and arginine (Arg-117). The structural determinants for binding malonyl CoA to FabD have been used by the inventor to design inhibitors of this enzyme. Accordingly, several new, small molecular-weight scaffolds have been identified. The structures of three promising scaffolds are presented below.

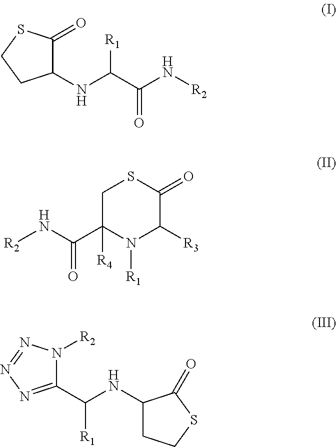

Further support that the thiolactone core is important for binding interactions within the active site of enzymes crucial for bacterial fatty acid synthesis comes from a high resolution X-ray structure of thiolactomycin bound to fatty acid synthase II (FAS-II). See Toohey, John I., Journal of Alzheimer's Disease 2007, 12(3), 241-243. As shown by this crystal structure, there is a network of hydrogen bonds between the thiolactone carbonyl and active site histidine residues. Furthermore, the enol moiety forms multiple hydrogen bonds with surrounding water and the carbonyl group of active site valine. Other interactions important for binding include hydrophobic and van der Waal interactions between the hydrophobic sulfur atom and the hydrophobic side chains of several active site residues, such as phenylalanine, alanine, proline and methionine. These results indicate that the geometry of the thiolactone scaffold is suitable for maximizing binding interactions with active site residues of enzymes important for fatty acid synthesis in bacteria. Given the central role of such enzymes for bacterial cell viability, inhibitors that incorporate the thiolactone scaffold should serve as potent antibiotic agents. The present invention also provides an in vitro enzyme assay for testing the effectiveness of the inventive compounds to inhibit the catalytic activity of FabD, as well as an in vivo cell assay for a cell viability study.

The present inventor has used x-ray data and mechanistic enzymology to design novel candidate inhibitors of FabD. Scheme 2 shows the enzymatic reaction sequence for FabD. As Scheme 2 illustrates, the first step of the reaction involves the cleavage of malonyl CoA by a nucleophilic residue in the active site of FabD (Ser 92). The subsequent step of this enzymatic reaction involves a transesterification reaction between acyl carrier protein (ACP) and the malonic acid modified serine group of FabD. Transesterification results in the formation of malonyl-ACP with the regeneration of CoA. Thus, suitable inhibitors of FabD, are molecular scaffolds that maintain the crucial salt bridge and hydrogen bonding interactions with active site residues of the protein and have in addition an acylating unit, such as a thiolactone in their chemical structure. Illustrative of the class of FabD inhibitors, are compounds that include a thiolactone scaffold (I and III) or a 6-oxothiomorpholino scaffold (II) (Scheme 2).

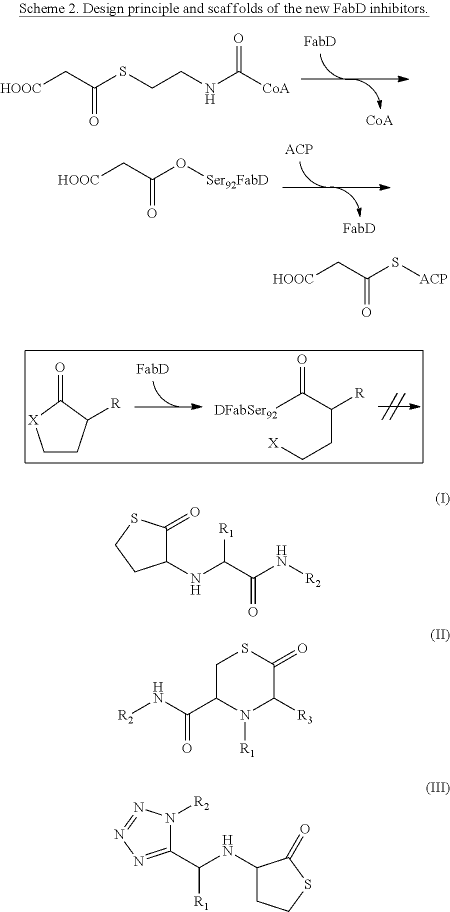

Compounds belonging to either the thiolactone or thiomorpholino class were designed to act as a warhead against active site nucleophiles of proteases, e.g., by reacting with active site serines or cysteines, as shown in Scheme 2. Synthesis of the illustrative inhibitor scaffolds were carried out using a multi-component reaction (MCR) methodology, that permits the rapid generation of a large number of compounds. See Doemling, A., Chem. Rev. 2006, 126, 17. For instance, reacting 10³ commercial aldehydes and ketones and 10³ amine-derived isocyanides results in the synthesis of a four million-compound library, which includes different isomers of the inventive scaffolds.

One advantage of MCR as a synthetic tool, is its superiority to sequential synthesis, in terms of ease of performance and available chemical space. Additionally, the reaction is stereoselective, allowing all diastereomers to be represented in the final chemical library. For instance, the end-on gamma-thiolactone scaffold (I) is accessible using a chiral homocysteine, an aldehyde or a ketone and an isocyanide. Structurally diverse derivatives of the γ-thiolactone can be prepared by the appropriate choice of the aldehyde/ketone or isocyanide that are readily available or synthetically accessible as starting materials.

Compounds synthesized using MCR, as described above, were tested for their ability to inhibit FabD using an in vitro inhibition assay. Briefly, FabD and holo-ACP was cloned, and expressed in E. coli according to the protocol of Molnos et al., Anal. Biochem. 2003, 319, 171. The proteins were purified using the ACTA protein purification system. The inhibition study was carried out according to the protocol of Miossec et al., American Society for Microbiology 104th General Meeting, New Orleans, 2004 (LA. Abstract: K-001), and was performed in a final volume of 100 μl in 96-well microtiter plates (IEA/RIA black, flat bottom, half area plates, Costar).

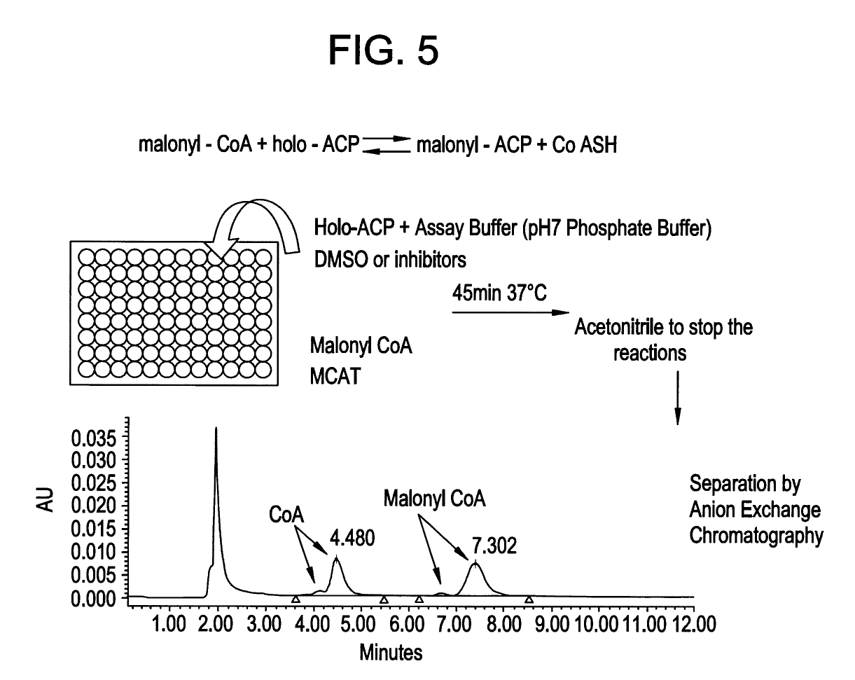

Purified E. coli FabD was diluted in the enzyme dilution buffer (100 mM phosphate buffer pH 7, DTT 1.5 mM, BSA 1 mg/ml [30 ml of Assay buffer+30 mg of BSA]) at ten times the final concentration (0.75 ng/ml). Malonyl-CoA (10 mM, Sigma) was diluted to ten times the final concentration (25 uM) with distilled H₂O. Holo-ACP was prepared to 874 μM. In a 96-well microplate, 5 ul of 874 μM Holo-ACP and 70 ul of assay buffer (100 mM phosphate buffer pH7, DTT 1.5 mM) were added and the resultant solution preincubated for 30 minutes at 37° C. by spinning at an orbital speed of 700 revolutions/minute. Five microliters of a solution of the FabD inhibitor was then added to the reaction mixture, followed by the addition of 10 μl of a 250 μM solution of malonyl-CoA. The enzyme reaction was initiated by the addition of 10 μl of MCAT, which was incubated at 37° C. for 45 minutes in a orbital shaker maintained at an orbital speed 700 revolutions/minute. The reaction was terminated by the addition of 100 μl of acetonitrile. The reaction mixture was analyzed by anion exchange, high performance liquid chromatography, using a PL-SAX8u 4.6 mm×150 mm column (Polymer Laboratories) and UV-detection (CoA and malonyl-CoA are detected at 254 nm). The percent inhibition of FabD was calculated by integrating the area of the CoA peak in the chromatogram. In the presence of an active inhibitor the area under the CoA peak diminishes or disappears (See FIG. 5).

### Access to the Thiolactone and Thiomorpholino Scaffolds Using MCR Chemistry

Multi-component reactions are a facile and economic way to generate a diverse library of chemical compounds, because the reaction allows the formation of a key intermediate which rearranges into structurally diverse scaffolds depending on the nature of an acidic component in the reaction mixture. As noted above, MCR chemistry was employed for synthesis of FabD inhibitors of the invention, and these were screened for their ability to inhibit FabD using the in vitro enzyme assay.

To identify potent inhibitors of FabD, the present inventor employed a publicly available software suite, maintained by Chemaxon (see www.chemaxon.com), to generate a virtual library of compounds for scaffolds identified by Formulae I-III in Scheme 2. The in silico synthetic route allowed for the rapid generation of ˜1×10⁵ compounds per scaffold. The virtual compounds then were tested for their ability to interact with the enzyme in silico, by docking each structure into the active site of the enzyme. Docking was performed in a constrained mode, to model the reaction between the active site serine and the reactive functionality. The results from such a virtual screen were used to identify a representative group of molecules, which were synthesized and tested for enzymatic and antibacterial activity.

Illustrative compounds within each scaffold were synthesized using the classical Ugi reaction. A mechanistic hallmark of Ugi-type, isocyanide-based MCRs (IMCRs) is the formation of a reactive intermediate, the alpha-adduct (Scheme 3). This intermediate results from the addition of a Schiff's base and an acid anion to the isocyanide carbon. The formation of the alpha-adduct is a unique feature of the isocyanide functional group and is responsible for the large scaffold diversity that results from subsequent rearrangement reactions of the intermediate in the presence of nucleophiles.

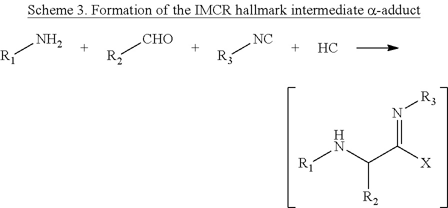

The design of new IMCR scaffolds was based on leveraging the acylation power of the α-adduct intermediate and the differential reactivity of nucleophilic starting materials that effect rearrangement of the intermediate. Compound classes in accord with this design principle, therefore, must have a functional group that is appropriate for the Ugi reaction and a suitably spaced reactive nucleophilic group that is capable of undergoing an intramolecular transacylation reaction. Pursuant to this synthetic methodology, an unprotected alpha amino acid was reacted with an appropriately substituted aldehyde or ketone and an isocyanide to give a cyclic intermediate 1′ (Scheme 4), which upon further reaction with a nucleophilic group, results in the formation of different molecular scaffolds as shown in Scheme 4.

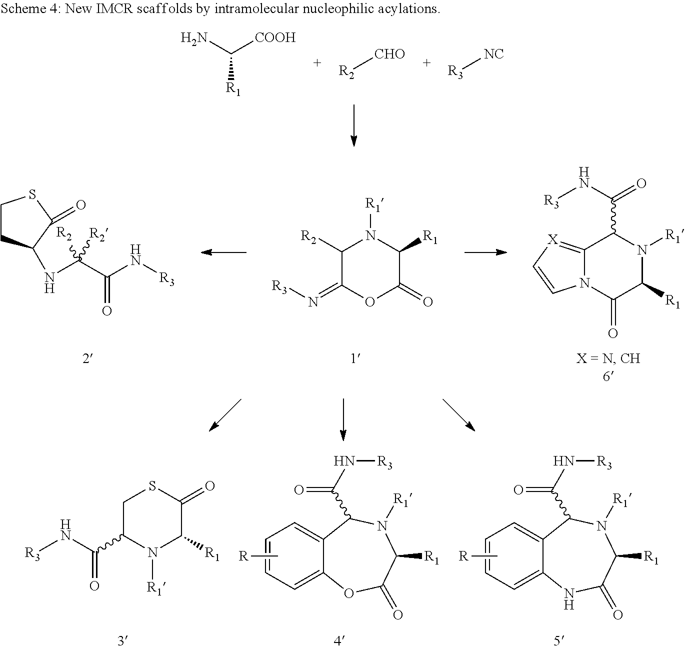

The chemical nature of substituent groups depends on the starting materials used in the Ugi reaction. Accordingly, the present invention provides compounds substituted with an alkyl group, an aryl group, a cycloalkyl group, a heteroaryl group, a heterocyclic group, an arylalkyl group or a heteroarylalkyl group.

“Alkyl” refers to straight, branched chain, or cyclic hydrocarbyl groups including from 1 to about 20 carbon atoms. Alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like Thus, alkyl groups include primary alkyl groups, secondary alkyl groups, and tertiary alkyl groups. Preferred alkyl groups include alkyl groups having from 1 to 10 carbon atoms while even more preferred such groups have from 1 to 5 carbon atoms.

The phrase “substituted alkyl” refers to alkyl substituted at 1 or more, e.g., 1, 2, 3, 4, 5, or even 6 positions, in which substituents are attached at any available atom to produce a stable compound, with substitution as described herein. “Optionally substituted alkyl” refers to alkyl or substituted alkyl.

The term “cycloalkyl” refers to saturated or unsaturated non-aromatic monocyclic, bicyclic or tricyclic carbon ring systems of 3-10, more preferably 3-6, ring members per ring, such as cyclopropyl, cyclopentyl, cyclohexyl, adamantyl, and the like.

The phrase “substituted cycloalkyl” refers to cycloalkyl substituted at 1 or more, e.g., 1, 2, 3, or even 4 positions, in which substituents are attached at any available atom to produce a stable compound, with substitution as described herein. “Optionally substituted cycloalkyl” refers to cycloalkyl or substituted cycloalkyl.

The term “aryl,” alone or in combination refers to a monocyclic or bicyclic ring system containing aromatic hydrocarbons such as phenyl or naphthyl, which may be optionally fused with a cycloalkyl of preferably 5-7, more preferably 5-6, ring members.

A “substituted aryl” is an aryl that is independently substituted with one or more, preferably 1, 2, 3, 4 or 5, also 1, 2, or 3 substituents, also 1 substituent, attached at any available atom to produce a stable compound, wherein the substituents are as described herein. “Optionally substituted aryl” refers to aryl or substituted aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl.

“Heterocycloalkyl” means a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl.

“Heteroalkyl” means a saturated or unsaturated alkyl group having from 1 to about 20 carbon atoms, preferably 1 to 10 carbon atoms, more preferably 1 to 6 carbon atoms, even more preferably 1 to 3 carbon atoms, in which from 1 to 3 carbon atoms are replaced by heteroatoms of O, S or N. Heteroalkyl is also intended to include oxidized S or N, such as sulfinyl, sulfonyl and N-oxide of a tertiary ring nitrogen. The point of attachment of the heteroalkyl substituent is at an atom such that a stable compound is formed. Examples of heteroalkyl groups include, but are not limited to, N-alkylaminoalkyl (e.g., CH₃NHCH₂—), N,N-dialkylaminoalkyl (e.g., (CH₃)₂NCH₂—), and the like.

“Arylalkyl” refers to a moiety of structure —R^{a}—R^{b}, wherein R^{a} is optionally substituted alkylene and R^{b} is aryl, as define herein. “Optionally substituted arylalkyl” means arylalkyl or arylalkyl wherein the aryl functionality is substituted with 1 to 3 substituents, e.g., 1, 2 or 3 substituents, attached at any available atom to produce a stable compound, wherein the substituents are as described herein.

“Heteroarylalkyl” refers to a moiety of structure —R^{a}—R^{c}, wherein R^{a} is optionally substituted alkylene and R^{c} is heteroaryl, as define herein. “Optionally substituted heteroarylalkyl” means heteroarylalkyl or heteroarylalkyl wherein the heteroaryl functionality is substituted with 1 to 3 substituents, e.g., 1, 2 or 3 substituents, attached at any available atom to produce a stable compound, wherein the substituents are as described herein.

The term “heterocycle” refers to monocyclic, bicyclic, tricyclic, or polycyclic systems, which are either unsaturated or aromatic and which contains from 1 to 4 heteroatoms, independently selected from nitrogen, oxygen and sulfur, wherein the nitrogen and sulfur heteroatoms are optionally oxidized and the nitrogen heteroatom optionally quaternized, including bicyclic, and tricyclic ring systems. The bicyclic or tricyclic ring systems may be spiro-fused. The bicyclic and tricyclic ring systems may encompass a heterocycle or heteroaryl fused to a benzene ring. The heterocycle may be attached via any heteroatom or carbon atom.

Scheme 5 illustrates that the structural fate of intermediate 1′ depends on the chemical nature of the nucleophile. Thus, when homocysteine is used as the alpha amino acid, an end-on thio-γ lactone 8′. In contrast, the use of a mercaptoaldehyde as the source of the nucleophilic sulfhydryl group results in the formation of a thiomorpholine derivative 9′ (Scheme 5). In scaffold 8′, the acylating unit is an end-on thiolactone that is easily accessible to nucleophiles within the protein's active site. The acylating thiolactone moiety in scaffold 9′ is hidden, however, and less accessible to nucleophiles. Thus, compounds belonging to this scaffold class are expected to show differential acylation of active site residues based on accessibility of such residues to the acylating thiolactone moiety.

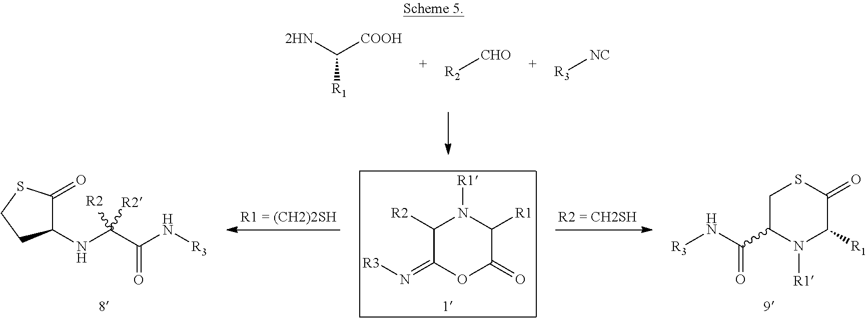

Exemplars of compounds belonging to the thiolactone and thiomorpholine class are shown below (Tables 1 and 2, respectively). Computational modeling and ab initio calculations of representative compounds made using commercially available starting materials reveal that most of the reaction products are “drug like,” obeying the Pfizer rules.

TABLE 1
Representative structures of thiolactone scaffold

| | Yield | Diastreomeric |
|---|---|---|
| Structure | [%] | ratio |
| 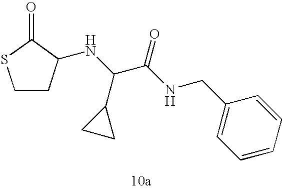 | 77 | 77:23 |
| 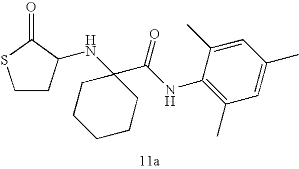 | 33 | — |
| 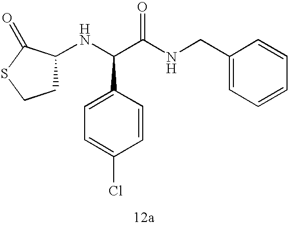 | — | — |
| 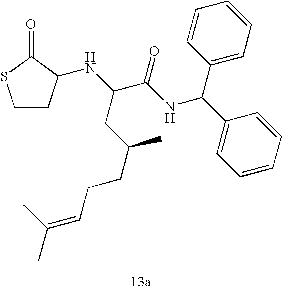 | 43 | nd |
| 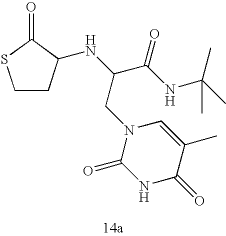 | 45 | 81:19 |
| 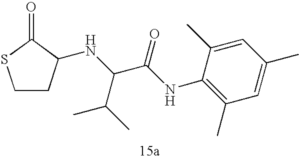 | 72 | nd |
| 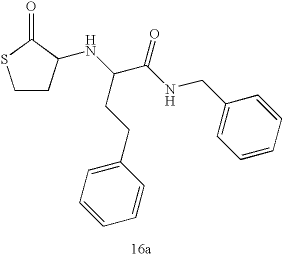 | 73 | nd |
| 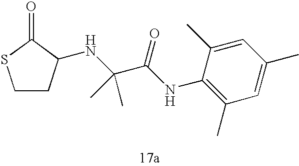 | 53 | — |
| 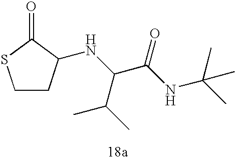 | 47 | 88:12 |
| 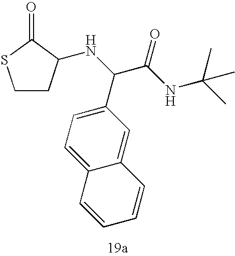 | 53 | 65:35 |
| 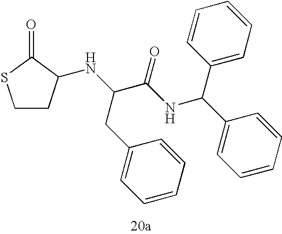 | 34 | 72:28 |

TABLE 2
Representative structures and isolated yields of thiomorpholine scaffold

| | | Yield* | | | |
|---|---|---|---|---|---|
| | | [%] | | | yield |
| no | structure | (de) | no | structure | [%] |
| 6b | 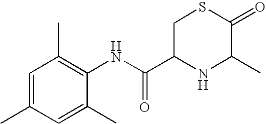 | 24 (78:22) | 9b | 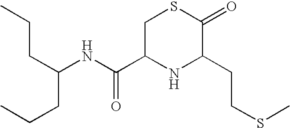 | 34 (96:4) |
| 7b | 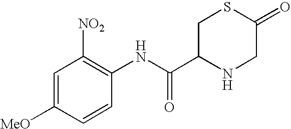 | 16 | 10b | 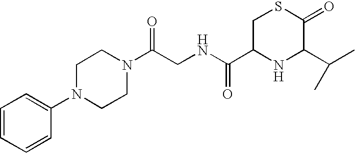 | 22 (73:27) |
| 8b | 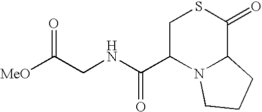 | 68 (76:24) | 21b | 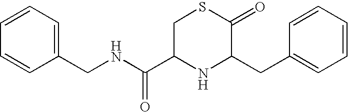 | 70 |
| 26b | 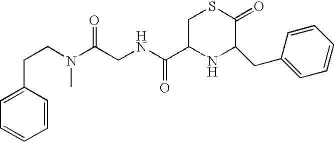 | 23 | 27b | 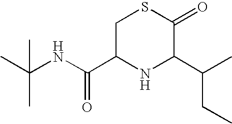 | 34 |
| 28b | 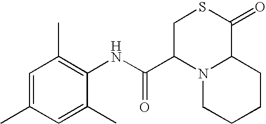 | 47 | 29b | 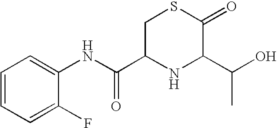 | 17 |
| 30b | 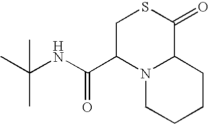 | 31 | | | |

*Yields are calculated on 50% conversion.

The three-component Ugi reaction also provides a facile method for synthesizing compounds that incorporate other molecular scaffolds. For example, commercially available salicylaldehyde has been used in the Ugi reaction to synthesize benzoxazepines (4′), while the benzodiazepine scaffold (5′) and imidazopyrazine scaffold (6′) compounds are readily synthesized using o-aminobenzaldehyde and 2-formylbenzimidazole respectively (Scheme 4).

### Cell-Based Screening Assay

Compounds belonging to the thiolactone and thiomorpholine class were tested for their ability to kill or inhibit the growth of bacterial cells in culture. Accordingly, a solution of FabD inhibitor of the invention was serially diluted into each well of a tissue culture plate so as to obtain a final inhibitor concentration in the range from about 100 to 1000 μg/ml (e.g., concentrations of 100, 200, 400, 600 and 800 ug/ml). Each well further contained 1000 μl Mueller-Hinton broth or Luria-Bertani broth. To initiate bacterial growth, five microliters of fresh bacterial culture was used as the inoculum DMSO was used as the control and the percent inhibition of bacterial growth was determined spectrophotometrically by measuring the optical density (OD) of the culture at 600 nm using a microtiter plate reader.

Initial antibacterial studies were performed using representative Gram positive and Gram negative bacteria, such as S. aureus and E. coli, respectively. The toxicity to mammalian cells was tested in vitro for compounds that display potent antibacterial activity using routine laboratory methods.

### Therapeutic Uses of Compounds of Formulae I-III

Use as an Antibiotic Agent

Compounds of this invention inhibit FabD, an enzyme that plays an important role in bacterial fatty acid synthesis. For example, three representative thiolactone derivatives, namely, compounds 12a, 13a and 15a (Table 1), were assayed for their ability to inhibit FabD according to the protocol disclosed by Molnos et al., supra. These representative compounds were found to be potent inhibitors of this enzyme. In one aspect, therefore, the inventive compounds can be used for treating bacterial infections in a subject. In the context of this invention, the terms “treat”, “treating,” and “treatment” refer to the amelioration or eradication of a bacterial infection. In certain embodiments, such terms refer to minimizing the systemic spread or worsening of infection resulting from the administration of compounds in accordance with this invention.

Accordingly, the invention provides formulations of compounds belonging to Formulae I-III, respectively, as potent selective inhibitors of bacterial infection. Such inhibition is reflected in various physiological and biochemical indicia, such as a decrease serum bacterial count, a lowering of body temperature during systemic infection, a lowering of the total white blood cell (WBC) count and improved wound healing when infection is localized.

Because compounds of this invention are selective inhibitors of bacterial growth, the amount of compound that results in greater than 95% inhibition of bacterial growth in vitro, can be used in determining an effective dose (“therapeutic dose”) in vivo, pursuant to conventional pharmaceutical practice. Thus, a pharmaceutical formulation that contains an amount of compound that results in blood concentrations equivalent to those documented here, such as an amount that produces greater than about 95% decrease in bacterial titre, can be a reasonable starting point for dose-response studies of bacterial inhibition in vivo. The results from such studies can readily inform the production of a formulation that exhibits the desired therapeutic effect.

Compounds according to this invention, can be formulated with a pharmaceutically acceptable carrier, either as a prodrug or as a pharmaceutically acceptable salt, solvate, stereoisomer, or tautomer. The term “prodrug” denotes a derivative of a compound that can hydrolyze, oxidize, or otherwise react under biological conditions, in vitro or in vivo, to provide an active compound, particularly a compound of the invention. Examples of prodrugs include but are not limited to biohydrolyzable groups such as biohydrolyzable amides, biohydrolyzable esters, biohydrolyzable carbamates, biohydrolyzable carbonates, biohydrolyzable ureides, and biohydrolyzable phosphate analogues (e.g., monophosphate, diphosphate or triphosphate). Prodrugs typically can be prepared using well-known methods, such as those described by BURGER'S MEDICINAL CHEMISTRY AND DRUG DISCOVERY 6^{th} ed. (Wiley, 2001) and DESIGN AND APPLICATION OF PRODRUGS (Harwood Academic Publishers Gmbh, 1985).

Although, the polarity of the thiolactone moiety for Formula I-III compounds, should promote their dissolution in aqueous carrier solvents, such as saline, the chemical nature and the number of functional substituent groups present on the thiolactone ring system will dictate the final composition of the formulation. For example, derivatives that have hydrophobic substituent groups will not readily dissolve in an aqueous medium. Formulations of such compounds, therefore, require the addition of a pharmaceutically acceptable hydrophobic solvent to the aqueous medium. Exemplars of pharmaceutically acceptable hydrophobic solvents include poly-alkylene glycol gelatin, gum arabic, lactose, starch, petroleum jelly and vegetable oil. Additional excipients, such as flavoring agents, preservatives, stabilizers, emulsifying agents, buffers and the like may be added in accordance with accepted practices of pharmaceutical formulation.

The FabD inhibitors can be administered topically, intravenously, intraperitoneally, orally, bucally, by insufflation, or by parenteral administration. Formulations suitable for oral administration can be in a solid or a liquid form, such as tablets, capsules, pills, powders, granules, and dragees. Solid oral formulations talc and/or carbohydrate carrier binder or the like, the carrier could be lactose and/or corn starch and/or potato starch. A syrup, elixir or the like could be used when a sweetened vehicle is desired.

Intravenous formulations suitable for treating a systemic infection preferably include oily or aqueous solutions of the inhibitors as well as suspensions, or emulsions. The compound is generally dispersed in a fluid carrier such as sterile physiological saline or 5% saline-dextrose solutions commonly used with injectables.

For topical applications, the inhibitor(s) can be suitably admixed in a pharmacologically inert topical carrier such as a gel, an ointment, a lotion or a cream. Such topical carriers include water, glycerol, alcohol, propylene glycol, fatty alcohols, triglycerides, fatty acid esters, or mineral oils. Other possible topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added if desired.

In accordance with the invention, treating bacterial infection entails identifying a subject suffering from a bacterial infection and then administering to the subject a therapeutic dose of a FabD inhibitor. Furthermore, the invention contemplates using inhibitors of FabD in order to gauge efficacy of such treatment. Pursuant to this aspect of the invention, efficacy is judged by comparing bacterial titres in two samples, each obtained at a different point or stage of treatment. A lower titer level in the sample obtained following the administration of FabD inhibitors is indicative of efficacy.

Use in Cancer Therapy

The thiolactone and thiomorpholine compounds were found by the present inventor to protect cells in normal tissue from the toxic side effects of ionizing radiation often used in cancer therapy. Such protection arises from the ability of the captioned compounds to inhibit the intracellular DNA repair and apoptosis targets p53 and caspase respectively.

Accordingly, formulations of compounds represented by Formulae I-III were tested for their ability to protect cells in vitro. Table 3 shows the structures of illustrative compounds. Of the compounds listed in this table, BEB 55 and BEB 59 are inhibitors of p53, while BEB 69, BEB 75, SK7 and SK61 were found to inhibit caspase.

TABLE 3

| | Structure | Mol. Wt | Net Wt (mg) |
|---|---|---|---|
| BEB55 | 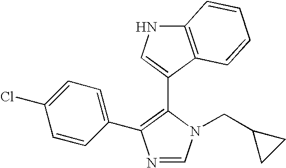 | 347.84 | 60.1 |
| BEB59 | 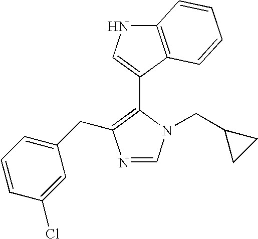 | 361.87 | 112.3 |
| SK7 (major isomer, racemic) | 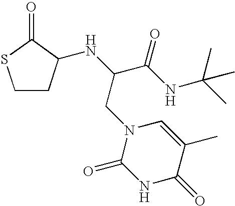 | 368.45 | 101 |
| SS61-1 (From D- alanine, major and the first isomer) | 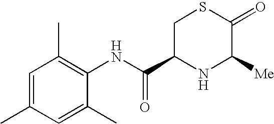 | 292.40 | 141 |
| SS61-2 (From D- alanine, minor and the second eluting isomer) | 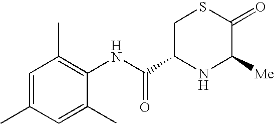 | 292.40 | 100 |
| SS61-3 (From L- alanine, major and the first eluting isomer) | 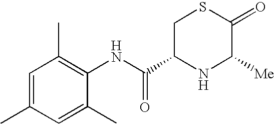 | 292.40 | 123 |
| SS61-4 (From L- alanine, minor and the second eluting isomer) | 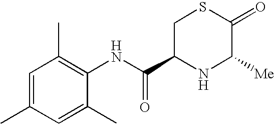 | 292.40 | 104 |
| BEB69 (major isomer, racemic) | 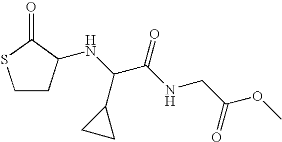 | 286.35 | 100 |
| BEB75-1 (From L- phenylglycine, major and the first eluting isomer) | 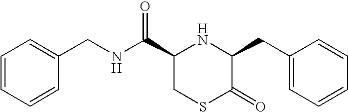 | 340.44 | 157 |
| BEB75-2 (From L- phenylglycine, minor and the second eluting isomer) | 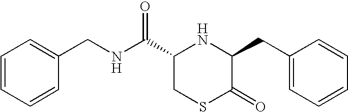 | 340.44 | 100 |
| BEB75-3 (From D-phenylglycine, major and the first eluting isomer) | 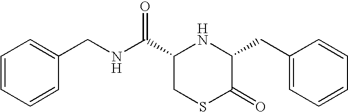 | 340.44 | 145 |
| BEB75-4 (From D-phenylglycine, minor and second eluting isomer) | 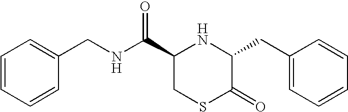 | 340.44 | 122 |

To measure the level of radioprotection for compounds in Table 3, a DMSO solution is made for each compound and added to 32D cl 3 cells, a mouse hematopoietic progenitor cell line at a final concentration of 10 μM. The terms “radioprotection” and “radioprotective” refer to the protective effect on normal cells exerted by the captioned compounds when such cells are exposed to ionizing radiation.

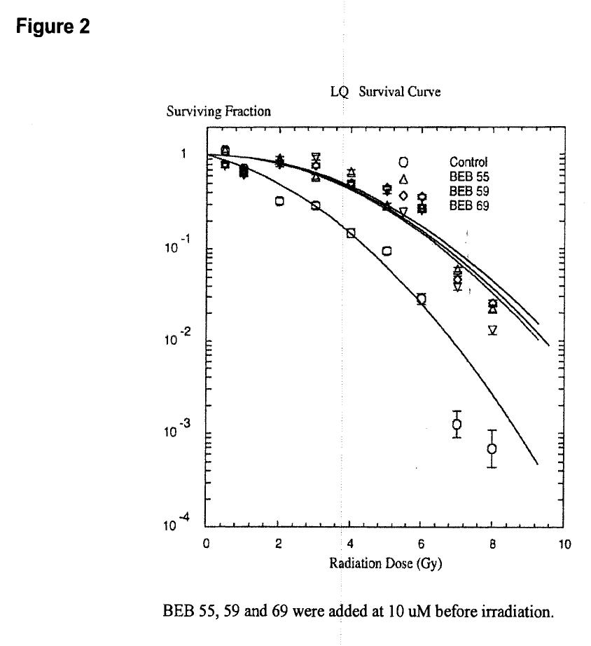

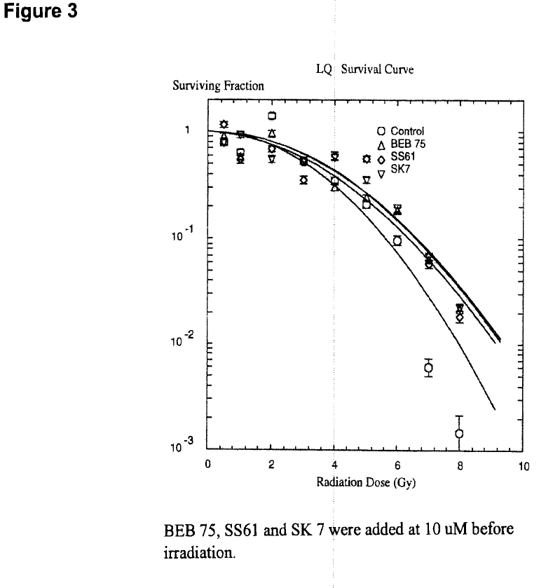

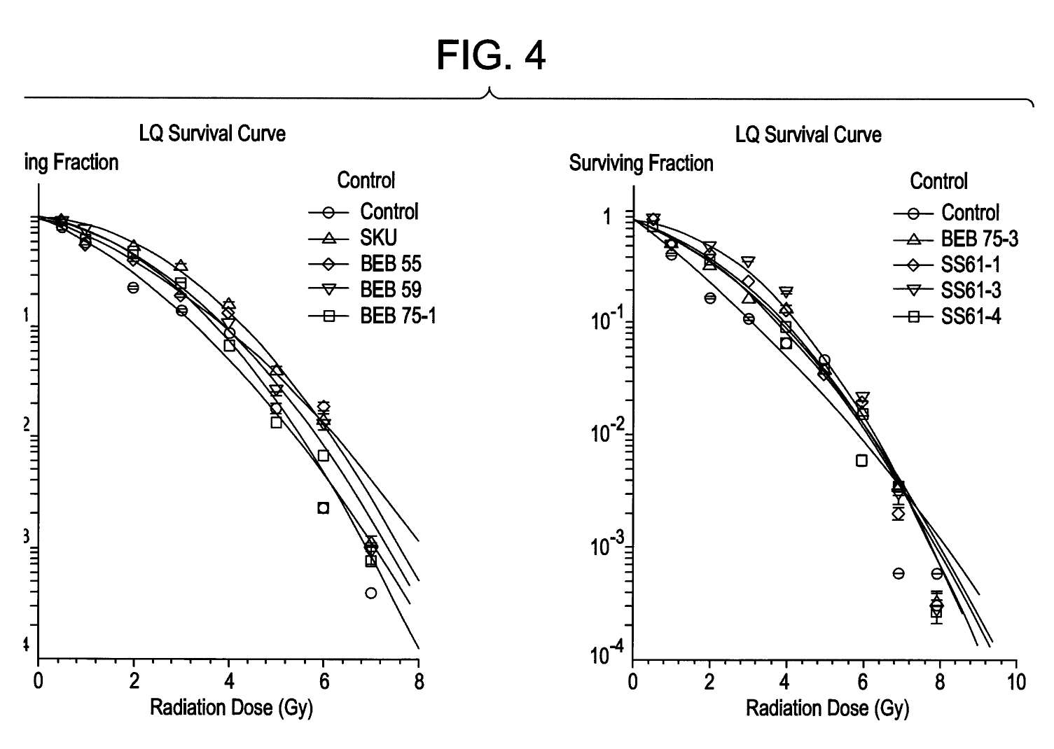

FIGS. 2-4 show the survival curves for 32D cl 3 cells in the presence of different compounds of the invention. A strong radioprotective effect is seen in cultures that include the captioned compounds. FIGS. 2 and 3 show cell survival curves when the captioned compounds were added to cell cultures before exposure to radiation. The survival curves in FIG. 4, however, show the radioprotective effect of the inventive compounds when delivered to cell cultures after exposure to ionizing radiation. The increased shoulder on the survival curves evident in FIGS. 2-4 demonstrates that the compounds mitigate the harmful effects of radiation. Moreover, the extent of radioprotection in a culture that is pretreated with an inventive compound is similar to that observed in a culture in which such compound is added after exposure to radiation.

The extent of radioprotection afforded by the captioned compounds is determined by measuring the fraction of viable cells in a culture containing the captioned radioprotective agent. The fraction of viable cells is determined by back-extrapolating the linear portion of the shoulder width in a survival curve to the Y-axis. The intersection of this line with the Y-axis represents the fraction of cells surviving the harmful effects of radiation in the presence of a radioprotecting agent and is denoted as N. Table 4 shows the N-values for cells incubated with a representative group of compounds. Both BEB 75-1 and SK7 were found to exert a potent radioprotective effect on cells, with N-values at least 4-fold greater than control. The radioprotective effect of these two compounds is due to their ability to inhibit caspase. Moreover, Table 4 indicates that both scaffolds are equally potent at protecting cells from radiation, because of the similar N-value for BEB 75-1 and SK-7.

TABLE 4

| Compound | [Do (Gy) | Ñ |
|---|---|---|
| 32D cl 3 | 1.1 ± 0.1 | 2.7 + 0.9 |
| SK7 | 1.1 ± 0.1 | 8.9 + 6.7 |
| BEB 55 | 1.1 ± 0.1 | 3.5 + 1.5 |
| BEB 59 | 1.1 ± 0.1 | 5.9 + 0.3 |
| BEB 75-1 | 0.9 ± 0.1 | 8.2 + 4.3 |

### Synthesis

General procedure for preparation of compounds 10a-20a: 2 mmol (270 mg) Homocycsteine is solubilized in 10 ml trifluorethanol (TFE) and cooled under nitrogen to −20° C. Solutions of 2 mmol isocyanide and 2 mmol aldehyde in 5 ml trifluoroethanol are added simultaneously drop wise using a syringe. The reaction mixture is stirred for 1 h in the cold and allowed to warm to room temperature and stirred over night. The solvent is evaporated and the residue is dissolved in ethyl acetate and extracted with water, and brine. The organic layer is dried over magnesium sulfate and concentrated. In most cases the product can be crystallized from ethyl acetate to yield the major diastereomer. In other cases the crude product is purified by column chromatography on silica gel with heptane/ethyl acetate elution gradient from 3/1 to 1/2.

N-Benzyl-2-cyclopropyl-2-(2-oxo-tetrahydro-thiophen-3-ylamino)-acetamide (10a). crystalline solid; yield 470 mg (77%) as a mixture of diastereomers (77:23); ¹H-NMR for the major diastereomer (CDCl₃, 600 MHz): δ=0.38 (m, 1H), 0.56-0.65 (m, 3H), 0.95-0.97 (m, 1H), 1.87 (m, 2H), 2.42-2.45 (m, 1H), 2.52 (d, 1H), 3.16-3.18 (m, 2H), 3.41-3.44 (m, 1H), 4.40-4.49 (m, 2H), 7.26-7.34 (m, 5H), 7.46 (br, 1H); ¹³C-NMR (CDCl₃, 150 MHz): δ=2.7, 3.1, 14.9, 26.4, 30.2, 32.3, 36.3, 65.3, 126.6, 126.9, 127.8, 137.6, 172.4, 207.0; HPLC-MS (ESI-TOF): rₜ=7.59 min m/z=305 [M+H]⁺; HRMS (ESI-TOF) m/z calcd for C₁₆H₂₀N₂O₂S [M+Na]⁺ 327.1143, found 327.1136.

1-(2-Oxo-tetrahydro-thiophen-3-ylamino)-cyclohexanecarboxylic acid (2,4,6-trimethyl-phenyl)-amide (11a). Crystalline solid; yield 236 mg (33%); ¹H-NMR (CDCl₃, 600 MHz): δ=1.34-1.42 (m, 2H), 1.47-1.65 (m, 1H), 1.67-1.78 (m, 4H), 1.87-1.88 (m, 1H), 1.96-2.16 (m, 2H), 2.20-2.24 (s, 6H and m, 2H), 2.27 (s, 3H), 2.67-2.71 (m, 1H), 3.16-3.23 (m, 2H), 3.49-3.52 (m, 1H), 6.84 (s, 2H), 8.83 (s, 1H); ¹³C-NMR (CDCl₃, 150 MHz): δ=18.4, 20.7, 21.2, 21.4, 22.8, 24.9, 26.7, 30.1, 32.5, 32.9, 34.5, 61.9, 63.4, 128.7, 128.9, 131.5, 134.6, 136.1, 175.0,209.0; HPLC-MS (ESI-TOF): rₜ=11.68 min m/z=361 [M+H]⁺; HRMS (ESI-TOF) m/z calcd for C₂₀H₂₈N₂O₂S [M]⁺360.1871 found 360.1871.

2-(4-Chloro-phenyl)-2-(2-oxotetrahydrothiophen-3-ylamino)-N-(1-propylbutyl)acetamide (12a). C₁₉H₂₇ClN₂O₂S; MW 382.94; Yield: 115 mg (30%) [diasteriomeric ratio 85:15]; HRMS found: m/z: 383.1742 [M+H]⁺, 405.1573 [M+Na]⁺; ¹H-NMR for the major isomer (CDCl₃, 600 MHz): δ=0.87-0.93 (m, 6H), 1.20-1.42 (m, 8H), 1.94-1.96 (m, 1H), 2.41-2.46 (m, 1H), 3.17-3.23 (m, 2H), 3.30-3.33 (m, 1H), 3.86-3.88 (m, 1H), 4.45 (brs, 1H), 6.38 (d, J=8.88 Hz, 1H), 7.30-7.33 (m, 4H). ¹³C-NMR (CDCl₃, 100 MHz): δ=13.75, 19.01, 27.48, 30.40, 37.18, 37.31, 48.93, 64.72, 65.47, 116.12, 128.60, 129.11, 134.32, 137.61, 170.59, 207.77.].

(S)-4,8-Dimethyl-2-(2-oxo-tetrahydro-thiophen-3-ylamino)-non-7-enoic acid benzhydryl-amide (13a). yellow oil; yield 396 mg (43%) as a mixture of diastereomers; 1H-NMR of the mixture of diasteromers (CDCl₃, 600 MHz): δ=0.91-0.96 (m, 3H), 1.23-1.25 (m, 1H), 1.37-1.60 (m, 2H), 1.66-1.74 (m, 6H), 1.79 (s, 3H), 1.81-1.92 (m, 2H). 1.94-2.02 (m, 2H), 2.36-2.38 (m, 1H), 3.03-3.06 (m, 2H), 3.24-3.28 (m, 2H), 5.05-5.08 (m, 1H), 6.22-6.26 (m, 1H), 7.19-7.46 (m, 10H), 8.11-8.24 (m, 1H); ¹³C-NMR (CDCl₃, 150 MHz): δ=17.71, 17.74, 19.0, 20.1, 25.2, 25.4, 25.76, 25.77, 27.0, 27.1, 29.2, 29.4, 29.5, 31.2, 31.3, 33.2, 36.4, 37.5, 41.0, 41.9, 42.2, 56.2, 56.39, 56.43, 59.3, 59.4, 66.56, 66.61, 124.5, 124.6, 126.9, 127.2, 127.3, 127.4, 127.5, 127.6,127.8, 128.58, 128.61, 128.65, 128.8, 131.4, 131.5, 141.7, 141.92, 141.96, 173.6, 173.9, 207.73, 207.77; carbon signals are doubled as a mixture of diastereomers; HPLC-MS (ESI-TOF): rₜ=12.73 min m/z=465 [M+H]⁺; HRMS (ESI-TOF) m/z calcd for C₂₈H₃₆N₂O₂S [M+Na]⁺ 464.6626, found 465.2765.

N-tert-Butyl-3-(5-methyl-2,4-dioxo-3,4-dihydro-2H-pyrimidin-1-yl)-2-(2-oxo-tetrahydro-thiophen-3-ylamino)-propionamide (14a). Colorless oil; yield 330 mg (45%) as a mixture of diastereomers (81:19); ¹H-NMR for the major diasteriomer (CDCl₃, 600 MHz): δ=1.34 (s, 9H), 1.88 (s, 3H), 1.90-2.03 (m, 2H), 2.44-2.50 (m, 1H), 3.21-3.27 (m, 2H), 3.32-3.34 (m, 1H), 3.39-3.44 (m, 1H), 3.96-4.00 (m, 1H), 4.09-4.12 (m, 1H), 7.1 (s, 1H), 7.57 (s, 1H), 9.73 (br, 1H); ¹³C-NMR (CDCl₃, 150 MHz): δ=12.2, 27.3, 28.5, 28.6, 30.9, 50.9, 51.1, 51.2, 61.8, 66.8, 111.0, 141.4, 152.8, 164.3, 170.2, 207.7; HPLC-MS (ESI-TOF): rₜ=8.87 min m/z=369 [M+H]⁺; HRMS (ESI-TOF) m/z calcd for C₁₆H₂₄N₄O₄S [M+Na]⁺ 339.1416, found 391.1397.

3-Methyl-2-(2-oxo-tetrahydro-thiophen-3-ylamino)-N-(2,4,6-trimethyl-phenyl)-butyramide (15a). Colorless solid; yield 160 mg (24 %) only the major diastereomer was isolated as precipitate; ¹H-NMR for the major diastereomer (CDCl₃, 600 mHz): δ=1.00 (d, 3H), 1.07 (d, 3H), 1.77 (br, 1H), 1.95 (m. 1H), 2.16 (s, 6H), 2.24 (s, 3H), 2.31-2.36 (m, 1H), 2.67-2.70 (m, 1H), 3.20-3.25 (m, 3H), 3.47-3.50 (m, 1H), 6.86 (s, 2H), 8.74 (s, 1H); ¹³C-NMR (CDCl₃, 150 MHz): δ=17.5, 18.7, 19.9, 20.7, 26.9, 31.1, 31.9, 67.1, 67.2, 128.8, 131.3, 134.5, 136.6, 171.5, 207.4; HPLC-MS (ESI-TOF): rₜ=10.88 min m/z=335 [M+H]⁺; HRMS (ESI-TOF) m/z calcd for C₁₈H₂₆N₂O₂S [M+Na]⁺ 357.1613, found 357.1598.

N-Benzyl-2-(2-oxo-tetrahydro-thiophen-3-ylamino)-4-phenyl-butyramide (16a). Colorless solid; yield 540 mg (73%) only the major diastereomer was isolated as precipitate; ¹H-NMR for the major diasteriomer (CDCl₃, 600 mHz): δ=1.75-1.93 (m, 3H), 2.10-2.13 (m, 1H), 2.40-2.43 (m, 1H), 2.70-2.76 (m, 2H), 3.10-3.15 (m, 2H), 3.22-3.23 (m, 1H), 3.31-3.35 (m, 1H), 4.36-4.48 (m, 2H), 7.16-7.19 (m, 3H), 7.24-7.27 (m, 5H), 7.30-7.33 (m, 2H), 7.53-7.55 (m, 1H); ¹³C-NMR (CDCl₃, 150 MHz): δ=26.3, 30.5, 31.4, 34.5, 42.4, 59.9, 65.6, 125.3, 126.6, 126.9, 127.6, 127.7, 127.8, 137.5, 140.1, 172.9, 207.0; HPLC-MS (ESI-TOF): rₜ=10.71 min m/z=368 [M+H]⁺; HRMS (ESI-TOF) m/z calcd for C₂₁H₂₄N₂O₂S [M+Na]⁺ 391.1456, found 391.1449.

2-Methyl-2-(2-oxo-tetrahydro-thiophen-3-ylamino)-N-(2,4,6-trimethyl-phenyl)-propionamide (17a). Colorless oil; yield 340 mg (53%); ¹H-NMR (CDCl₃, 600 MHz): δ=1.47 (d, 6H, J=6.60 Hz), 1.97-2.04 (m, 2H), 2.14 (s, 6H), 2.25 (s, 3H), 2.71-2.73 (m, 1H), 3.21-3.27 (m, 2H), 3.54-3.57 (m, 1H), 6.86 (s, 2H), 8.92 (s, 1H); ¹³C-NMR (CDCl₃, 150 MHz): δ=18.5, 20.9, 26.4, 26.7, 26.8, 34.9, 59.8, 63.9, 128.9, 131.4, 134.8, 136.5, 174.9, 208.8; HPLC-MS (ESI-TOF): rₜ=10.40 min m/z=321 [M+H]⁺; HRMS (ESI-TOF) m/z calcd for C17H24N2O2S [M+H]⁺ 321.1637, found 321.1639.

N-tert-Butyl-3-methyl-2-(2-oxo-tetrahydro-thiophen-3-ylamino)-butyramide (18a). Colorless solid; yield 254 mg (47%) as a mixture of diastereomers (88:12); ¹H-NMR for the major diastereomer (CDCl₃, 600 MHz): δ=0.91 (d, 3H, J=6.96 Hz), 1.01 (d, 3H, J=6.96 Hz),1.35 (s, 9H), 1.87-1.94 (m, 1H), 2.11-2.16 (m, 1H), 2.53-2.57 (m, 1H), 2.85 (d, 1H, J=4.68 Hz), 3.19-3.32 (m, 3H), 6.98 (s, 1H); ¹³C-NMR (CDCl₃, 150 MHz): δ=17.3, 19.2, 27.4, 28.5, 31.3, 33.3, 50.4, 68.2, 68.6, 172.5, 206.9; HPLC-MS (ESI-TOF): rₜ=10.05 min m/z=273 [M+H]⁺; HRMS (ESI-TOF) m/z calcd for C₁₃H₂₄N₂O₂S [M+Na]⁺ 295.1456, found 295.1457.

N-tert-Butyl-2-naphthalen-2-yl-2-(2-oxo-tetrahydro-thiophen-3-ylamino)-acetamide (19a). Colorless solid; yield 380 mg (53%) as a mixture of diastereomers (65:35); ¹H-NMR of the mixture of diasteromers (CDCl₃, 600 MHz): δ=1.30 (s, 9H), 1.32 (s, 9H from minor isomer), 1.92-2.03 (m, 1H), 2.34-2.38 (m, 1H), 2.60-2.65 (m, 1H from minor isomer), 3.11-3.24 (m, 2H), 3.33-3.36 (m, 1H), 3.41-3.44 (m, 1H from minor isomer), 4.39 (s, 1H from minor isomer), 4.55 (s, 1H), 6.12 (s, 1H), 6.95 (s, 1H from minor isomer), 7.36-7.51 (m, 4H), 7.79-7.85 (m, 3H); ¹³C-NMR (CDCl₃, 150 MHz): δ=27.6, 28.7, 28.8, 32.2, 32.9, 51.1, 51.2, 65.5, 65.7, 66.7, 67.2, 124.7, 125.1, 126.2, 126.3, 126.36, 126.4, 126.5, 127.4, 127.70, 127.72, 128.02, 128.04, 128.92, 128.94, 133.1, 133.2, 133.3, 136.4, 136.6, 170.7, 170.9, 207.1, 208.1; carbon signals are doubled as a mixture of diastereomers; HPLC-MS (ESI-TOF): rₜ=11.13 min m/z=357 [M+H]⁺; HRMS (ESI-TOF) m/z calcd for C20H24N2O2S [M+Na]⁺ 379.1456, found 379.1468.

N-Benzhydryl-2-(2-oxo-tetrahydro-thiophen-3-ylamino)-3-phenyl-propionanide (20a). Colorless solid; yield 292 mg (34%) as a mixture of diastereomers (72:28); ¹H-NMR of the mixture of diasteromers (CDCl₃, 600 MHz): δ=1.48-1.54 (m, 1H), 1.62-1.69 (m, 1H), 2.14-2.19 (m, 1H), 2.23-2.27 (m, 1H from minor diasteromer), 2.83-2.86 (m, 1H), 2.98-3.07 (m, 2H), 3.17-3.25 (m, 1H), 3.31-3.34 (m, 1H), 3.48-3.51 (m, 1H), 3.57-3.58 (m, 1H from minor diastereomer), 6.28 (d, 1H, J=8.94 Hz), 7.15-7.36 (m, 15H), 8.39 (d, 1H, J=8.94 Hz); ¹³C-NMR (CDCl₃, 150 MHz): δ=26.9, 27.5, 31.1, 33.0, 39.2, 39.3, 56.4, 56.6, 62.3, 63.2, 66.9, 67.1, 127.1, 127.2, 127.3, 127.36, 127.37, 127.39, 127.45, 127.58, 127.61, 128.58, 128.62, 128.64, 128.74, 128.86, 128.88, 129.27, 129.45, 136.5, 137.2, 141.1, 141.7, 141.8, 172.2, 172.3, 206.7, 207.6; carbon signals are doubled as a mixture of diastereomers; HPLC-MS (ESI-TOF): rₜ=11.53 min m/z=431 [M+H]⁺; HRMS (ESI-TOF) m/z calcd for C₂₆H₂₆N₂O₂S [M+Na]⁺ 453.1613, found 453.1619.

General procedure for preparation of the thiomorpholine compounds: Amino acid (1 mmol) is dissolved in 10 ml trifluoroethanol at room temperature. 1 mmol Isocyanide and 0.5 mmol 2,5-dihydroxy-1,4-dithiane are added simultaneously to it. The reaction mixture is stirred over night at room temperature. The solvent is evaporated and the residue is dissolved in ethyl acetate and extracted with water and brine. The organic layer is dried over magnesium sulfate and concentrated. The crude product is purified by column chromatography on silica gel with petroleum ether/ethyl acetate, elution gradient from 2/1 to 1/2.

5-Methyl-6-oxo-thiomorpholine-3-carboxylic acid (2,4,6-trimethyl-phenyl)-amide (6b). Viscous oil; yield: 105 mg (12%) as a mixture of diastereomers (78:22); ¹H-NMR for the major diastereomer (CDCl₃, 600 MHz): δ=1.28 (s, 3H), 2.10 (s, 6H), 2.20 (s, 3H), 2.39 (brs, 1H), 3.31-3.36 (m, 1H), 3.54-3.59 (m, 2H), 3.85-3.86 (m, 1H), 6.84 (m, 2H); ¹³C-NMR (CDCl₃, 150 MHz): δ=15.5 18.1, 20.6, 30.8, 54.8, 55.4, 128.7, 130.3, 134.1, 136.8, 169.6, 202.1; HPLC-MS (ESI-TOF): rₜ=10.35 min m/z=293 [M+H]⁺; HRMS (ESI-TOF) m/z calcd for C₁₅H₂₀N₂O₂S [M+H]⁺ 292.1324, found 293.1321.

[(1-Oxo-hexahydro-pyrrolo[2,1-c][1,4]thiazine-4-carbonyl)-aminol-acetic acid methyl ester (8b). Crystalline solid; yield: 275 mg (34%) as a mixture of diastereomers (76:24); ¹H-NMR for the major diastereomer (CDCl₃, 600 MHz): δ=1.86-1.94 (m, 2H), 1.95-2.05 (m, 1H), 2.20-2.28 (m, 1H), 2.59-2.64 (m, 1H), 3.30-3.35 (m, 1H), 3.40 (d, 1H, J=12 Hz), 3.55-3.60 (m, 1H), 3.61-3.67 (m, 1H), 3.69-3.73 (m, 1H), 3.75 (s, 3H), 3.91-3.96 (m, 1H), 4.20-4.27 (m, 1H), 7.69 (brs, 1H); ¹³C-NMR (CDCl₃, 150 MHz): δ=21.9, 24.1, 29.7, 31.2, 40.8, 52.4, 52.7, 61.3, 65.3, 170.0, 171.7, 201.3; HPLC-MS (ESI-TOF): rₜ=8.45 min m/z=273 [M+H]⁺; HRMS (ESI-TOF) m/z calcd for C₁₁H₁₆N₂O₄S [M+Na]⁺ 295.0728, found: 295.0723.

5-(2-Methylsulfanyl-ethyl)-6-oxo-thiomorpholine-3-carboxylic acid (1-propyl-butyl)-amide (9b). Viscous oil; yield: 163 mg (16%) as a diastereomeric mixture (96:4); ¹H-NMR for the major diasteriomer (CDCl₃, 600 MHz): δ=0.92 (m, 6H), 1.23-1.43 (m, 6H), 1.45-1.54 (m, 2H), 1.65-1.73 (m, 1H), 2.05-2.22 (m, 4H), 2.29-2.36 (brs, 1H), 2.68-2.73 (m, 2H), 3.24-3.31 (m, 1H), 3.56-3.64 (m, 2H), 3.75-3.81 (m, 1H), 3.86-3.94 (m, 1H), 7.48 (brs, 1H); ¹³C-NMR (CDCl₃, 150 MHz): δ 14.0, 15.6, 19.1, 19.4, 28.1, 30.9, 31.3, 37.4, 37.5, 48.8, 54.9, 58.3, 170.9, 202.6; HPLC-MS (ESI-TOF): rₜ=11.27 min m/z=333 [M+H]⁺; HRMS (ESI-TOF) m/z calcd for C₁₅H₂₈N₂O₂S₂ [M+H]⁺ 333.1670, found 333.1663.

5-Isopropyl-6-oxo-thiomorpholine-3-carboxylic acid [2-oxo-2-(4-phenyl-piperazin-1-yl)-ethyl]-amide (10b). Viscous oil; yield: 126 mg (10%) as a mixture of diastereomers (73:27); ¹H-NMR for the diastereomeric mixture (CDCl₃, 600 MHz): δ=1.04 (d, 3H, J=7.2 Hz), 1.08 (d, 1H, J=6.6 Hz,from minor diast.), 1.12 (d, 1H, J=7.2 Hz, from minor diast.), 1.19 (d, 3H, J=6.6 Hz), 1.86-1.91 (m, 0.34H,from minor diast.), 2.18-2.34 (brs, 1H), 2.36-2.41 (m, 1H), 3.17-3.29 (m, 6H), 3.32 (dd, 1H, J=12 Hz & 14.4 Hz), 3.41 (dd, 0.30H, J=7.8 Hz & 12.6 Hz, from minor diast.), 3.56-3.62 (m, 4H), 3.72 (t, 0.71H, from minor diast.), 3.79-3.90 (m, 3H), 4.05 (dd, 0.38H, J=3.6 Hz & 17.4 Hz, from the minor diast.), 4.16 (dd, 1H, J=4.2 Hz & 10.2 Hz), 4.48 (brd, 0.36H, from minor diast.), 6.93-6.96 (m, 3H & 1H from minor diast.), 7.28-7.32 (m, 2H & 1H from minor diast.), 8.35 (brs, 0.31H, from minor diast.), 8.50 (brs, 1H); ¹³C-NMR (CDCl₃, 150 MHz): δ=17.1, 20.3, 27.4, 30.9, 41.4, 44.4, 49.4, 49.6, 54.9, 64.4, 116.8, 120.9, 129.3, 166.1, 172.2, 202.0; HPLC-MS (ESI-TOF): rₜ=10.29 min m/z=405 [M+H]⁺; HRMS (ESI-TOF) m/z calcd for C₂₀H₂₈N₄O₃S [M+H]⁺ 405.1960, found 405.1995.

5-Benzyl-6-oxo-thiomorpholine-3-carboxylic acid benzylamide (21b). Crystalline solid; yield: 180 mg (70%) as a mixture of diastereomers; ¹H-NMR for the major diastereomer (CDCl₃, 600 MHz): δ=2.31-2.34 (m, 1H), 2.42-2.48 (m, 1H), 3.28-3.31 (m, 1H), 3.35-3.36 (m, 1H), 3.38-3.40 (m, 1H), 3.53-3.68 (m, 1H), 3.70-3.75 (m, 2H). 4.17-4.21 (m, 1H), 6.89-7.32 (m, 11H); ¹³C-NMR (CDCl₃, 150 MHz): δ=30.9, 35.2, 42.5, 54.1, 61.6, 126.6, 127.1, 127.3, 128.2, 128.3, 129.1, 137.9, 171.1, 202.3; HPLC-MS (ESI-TOF): rₜ=11.61 min m/z=341 [M+H]⁺; HRMS (ESI-TOF) m/z calcd for Cl₉H₂₀N₂O₂S [M+H]⁺ 341.1324, found 341.0897.

5-Benzyl-6-oxo-thiomorpholine-3-carboxylic acid [(methyl-phenethyl-carbamoyl)-methyl]-amide (26b). Viscous oil; yield: 298 mg (23%) as a mixture of diastereomers (57:43); ¹H-NMR for the diastereomeric mixture (CDCl₃, 600 MHz): δ=2.20-2.41 (brs, 1H), 2.79-2.85 (m, 4H), 2.87 (t, 3H, J=7.8 Hz), 2.99 (s, 1.7H, from minor diast.), 3.26-3.37 (m, 3H), 3.46-3.69 (m, 8H), 3.76-3.79 (dd, 0.7H, J=6 Hz & 11.4 Hz, from minor diast.), 3.80-3.85 (m, 1H), 7.18-7.36 (m, 15H), 7.59 (brs, 0.66H, from the minor diast.), 7.64 (brs, 0.87H); ¹³C-NMR (CDCl₃, 150 MHz): δ=31.1, 33.8, 34.7, 35.5, 40.2, 40.8, 50.4, 50.7, 54.4, 61.6, 126.5, 126.9, 128.5, 128.6, 128.8, 128.9, 129.9, 137.8, 139.1, 167.4, 172.3, 202.4; HPLC-MS (ESI-TOF): rₜ=10.58 min m/z=426 [M+H]⁺; HRMS (ESI-TOF) m/z calcd for C₂₃H₂₇N₃O₃S [M+H]⁺ 426.1851, found 426.1837.

5-sec-Butyl-6-oxo-thiomorpholine-3-carboxylic acid tert-butylamide (27b). Viscous oil; yield: 212 mg (34%) as a mixture of diastereomers (73:27); ¹H-NMR for the diastereomeric mixture (CDCl₃, 600 MHz): δ=0.93 (d, 3H, J=5.88 Hz), 0.95-0.98 (m, 3H), 1.09 (d, 1.47H, J=6.78 Hz, from minor diast.), 1.38 (s, 9H), 1.40-1.60 (m, 2H), 2.08-2.21 (m, 2H), 3.15 (m, 0.37H, from minor diast.), 3.25-3.31 (m, 2H), 3.54-3.59 (m, 1H), 3.69-3.73 (m, 1H), 7.62 (brs, 1H); ¹³C-NMR (CDCl₃, 150 MHz): δ=11.9, 14.0, 16.9, 23.9, 27.3, 28.7, 30.9, 33.4, 33.7, 50.8, 54.7, 54.9, 61.9, 64.4, 170.8, 170.9, 202.4, 202.9; HPLC-MS (ESI-TOF): rₜ=10.92 min m/z=273 [M+H]⁺; HRMS (ESI-TOF) m/z calcd for C₁₃H₂₄N₂O₂S [M+H]⁺ 273.1637, found 273.1637.

1-Oxo-octahydro-pyrido[2,1-c][1,4]thiazine-4-carboxylic acid (2,4,6-trimethyl-phenyl)-amide (28b). Viscous oil; yield 466 mg (47%) as a mixture of diastereomers (85:15), ¹H-NMR for the major diastereomer (CDCl₃, 600 MHz): δ=1.37 (m, 1H), 1.53-1.66 (m, 1H), 1.70-1.90 (m, 3H), 1.99-2.30 (m, 14H), 2.74-2.85 (m, 1H), 3.01-3.11 (m, 1H), 3.35-3.47 (m, 1H), 3.57-3.78 (m, 3H), 6.90 (s, 2H), 9.00 (brs, 1H); ¹³C-NMR (CDCl₃, 150 MHz): 618.5, 20.9, 26.2, 27.1, 53.6, 64.5, 129.1, 130.9, 134.7, 136.9, 169.0, 200.3; ¹H-NMR for the minor diastereomer (CDCl₃, 600 MHz): δ=1.61-1.65 (m, 3H), 1.74-1.75 (m, 1H), 1.95-2.03 (m, 1H), 2.20 (s, 6H), 2.29 (s, 3H), 2.37 (brd, 1H, J=12.6 Hz), 2.85-2.93 (m, 2H), 3.37 (t, 1H, J=12 Hz), 3.55 (dd, 1H, J=12 Hz & 5.4 Hz), 3.66 (dd, 1H, J=14.4 Hz & 5.4 Hz), 3.91 (brs, 1H), 6.93 (s, 2H), 9.22 (s, 1H); ¹³C-NMR (CDCl₃, 150 MHz): δ=18.4, 19.6, 20.9, 25.2, 26.6, 53.5, 60.0, 65.6, 129.1, 130.7, 134.2, 137.1, 169.0, 201.2; HPLC-MS (ESI-TOF): rₜ=11.28 min m/z=333 [M+H]⁺; HRMS (ESI-TOF) m/z calcd for C₁₈H₂₄N₂O₂S [M+Na]⁺ 355.1456, found 355.1447.

5-(1-Hydroxy-ethyl)-6-oxo-thiomorpholine-3-carboxylic acid (2-fluoro-phenyl)-anide (29b). Viscous oil; yield: 107 mg (12%) as a mixture of diastereomers (81:19); ¹H-NMR for the major diastereomer (CDCl₃, 600 MHz): δ=1.46 (d, 3H, J=12 Hz), 2.37 (brs, 1H), 2.96 (t, 1H, J=12 Hz), 3.31-3.35 (m, 2H), 3.69 (dd, 1H, J=18 Hz & 6 Hz), 4.00-4.05 (m, 1H), 4.63-4.67 (m, 1H), 7.06-7.17 (m, 3H), 8.41 (m, 1H), 10.03 (brs, 1H); ¹³C-NMR (CDCl₃, 150 MHz): δ=18.6, 30.7, 55.6, 63.4, 64.5, 115.9, 120.8, 124.7, 125.8, 169.9, 203.3; HPLC-MS (ESI-TOF): rₜ=9.42 min m/z=299 [M+H]⁺; HRMS (ESI-TOF) m/z calcd for C₁₃H₁₅FN₂O₃S [M+H]⁺ 299.0866, found 299.0850.

1-Oxo-octahydro-pyrido[2,1-c][1,4]thiazine-4-carboxylic acid tert-butylamide (30b). Viscous oil; yield 254 mg (31%) as amixture of diastereomers (64:36); ¹H-NMR for the major diastereomer (CDCl₃, 600 MHz): δ=1.39 (s, 9H), 1.50-1.56 (m, 3H), 1.61-1.63 (m, 1H), 1.70 (brd, 1H, J=9.6 Hz), 2.33-2.35 (brd, 1H, J=2.2 Hz), 2.73-2.80 (m, 2H), 3.24-3.25 (m, 2H), 3.57 (d, 1H, J=3 Hz), 3.75 (m, 1H), 7.76 (brs, 1H); ¹³C-NMR (CDCl₃, 150 MHz): δ=19.2, 25.3, 26.7, 28.7, 30.9, 50.4, 52.9, 59.8, 65.4, 169.7, 201.6; ¹H-NMR for the minor diastereomer (CDCl₃, 600 MHz): δ=1.37 (s, 9H), 1.42-1.40 (m, 1H), 1.57-1.75 (m, 3H), 1.78-1.99 (m, 2H), 2.66 (m, 1H), 2.87-2.90 (m, 1H), 3.41-3.56 (m, 3H), 3.63-3.66 (m, 1H), 7.43 (brs, 1H); ¹³C-NMR (CDCl₃, 150 MHz): δ=22.6, 26.0, 27.0, 27.7, 28.6, 50.9, 63.8, 66.4, 169.2, 200.3; HPLC-MS (ESI-TOF): rₜ=10.69 min m/z=271 [M+H]⁺; HRMS (ESI-TOF) m/z calcd for C₁₃H₂₂N₂O₂S [M+Na]⁺ 293.1300, found 293.1285.

What is claimed is:

1. A method for treating bacterial infection in a subject in need thereof, comprising (A) identifying said subject and then (B) administering to the subject a formulation comprising a therapeutic dose of a compound according to one of Formula I, Formula II, and Formula III: 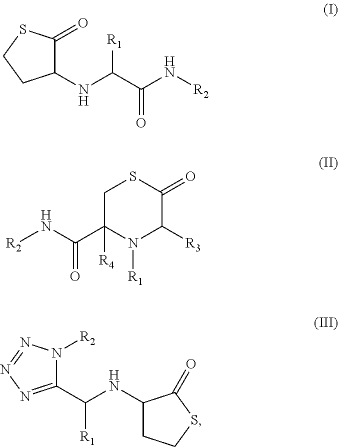
wherein:
R₁ is selected from the group consisting of (C₁-C₆)alkyl, (C₃-C₆)aryl, (C₃-C₆)heteroaryl, arylakyl, and heteroarylalkyl;
R₂ is selected from the group consisting of (C₁-C₆)alkyl, (C₃-C₆)aryl, and (C₃-C₆)heteroaryl, arylakyl, heterocycloalkyl, and heteroarylalkyl;
R₃ is selected from the group consisting of (C₁-C₆)alkyl, (C₃-C₆)aryl, and (C₃-C₆)heteroaryl, arylakyl, heterocycloalkyl, and heteroarylalkyl; and
R₄ is selected from the group consisting of (C₁-C₆)alkyl and heterocycle.

2. The method of claim 1, wherein the bacterial infection is a systemic infection.

3. A method for protecting normal tissue from the toxic effects of ionizing radiation, comprising (A) administering to a subject, who is undergoing radiation chemotherapy, a formulation comprising a therapeutic dose of a compound according to one of Formula I, Formula II, and Formula III: 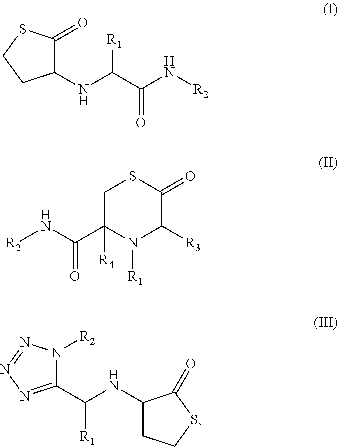
wherein:
R₁ is selected from the group consisting of (C₁-C₆)alkyl, (C₃-C₆)aryl, (C₃-C₆)heteroaryl, arylakyl, and heteroarylalkyl;
R₂ is selected from the group consisting of (C₁-C₆)alkyl, (C₃-C₆)aryl, and (C₃-C₆)heteroaryl, arylakyl, heterocycloalkyl, and heteroarylalkyl;
R₃ is selected from the group consisting of (C₁-C₆)alkyl, (C₃-C₆)aryl, and (C₃-C₆)heteroaryl, arylakyl, heterocycloalkyl, and heteroarylalkyl; and
R₄ is selected from the group consisting of (C₁-C₆)alkyl and heterocycle.

4. A pharmaceutical composition comprising (A) a therapeutically effective dose of a compound according to any one of Formulae I-III 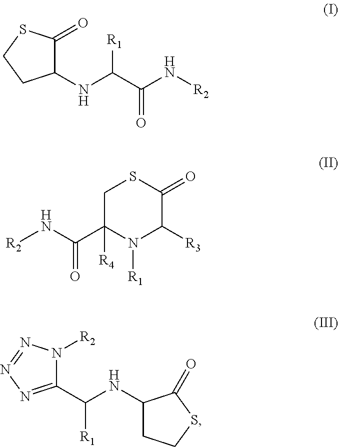
or a pharmaceutically acceptable salt, solvate, stereoisomer, tautomer, or prodrug of said compound, wherein:
R₁ is selected from the group consisting of (C₁-C₆)alkyl, (C₃-C₆)aryl, (C₃-C₆)heteroaryl, arylakyl, and heteroarylalkyl;
R₂ is selected from the group consisting of (C₁-C₆)alkyl, (C₃-C₆)aryl, and (C₃-C₆)heteroaryl, arylakyl, heterocycloalkyl, and heteroarylalkyl;
R₃ is selected from the group consisting of (C₁-C₆)alkyl, (C₃-C₆)aryl, and (C₃-C_{C}₆)heteroaryl, arylakyl, heterocycloalkyl, and heteroarylalkyl; and
R₄ is selected from the group consisting of (C₁-C₆)alkyl, and heterocycle;
and (B) a pharmaceutically acceptable carrier thereof
